(19) United States
(12) Patent Application Publication
(10) Pub. No.: US 2005/0036959 A1
(43) Pub. Date: Feb. 17, 2005

(54) Rapid temporary tooth whitening composition

(76) Inventors: Sayed Ibrahim, Somerset, NJ (US); Suman K. Chopra, Dayton, NJ (US); Michael Prencipe, West Windsor, NJ (US)

Correspondence Address: Colgate-Palmolive Company;909 River Road, P.O. Box 1343, Piscataway, NJ, 08855-1343, US

(21) Appl. No.: 10/641,963

(22) Filed: Aug. 15, 2003

(51) Int. Cl. A61K 7/16; A61K 7/24

(52) U.S. Cl. 424/55

(57) ABSTRACT

A composition for the rapid, temporary whitening of teeth comprising a combination of an adhesive material and hydroxyapatite.

### BACKGROUND OF THE INVENTION

1. Field of the Invention

This invention is directed to a composition of cosmetically altering the appearance of teeth and more particularly to a composition which provides rapid temporary cosmetic tooth whitening.

2. The Prior Art

Many substances such as tea and coffee that a person confronts or comes in contact with on a daily basis can “stain” or reduce the “whiteness” of one's teeth. Consumers consider clean, white teeth to be aesthetically desirable. Dull looking, stained teeth are objectionable to most people both on the basis of cosmetic appearance and also socially as an indication of poor oral hygiene. One whose teeth are white may enjoy more personal confidence and satisfaction and may even enjoy greater social acceptance.

Dental gel compositions containing active oxygen liberating ingredients such as hydrogen peroxide, urea peroxide, percarbonates and perborates of alkali and alkaline earth metals have been disclosed in the prior art for whitening teeth. Such gel compositions although effective for whitening teeth require multiple applications of the gel over a period of one to two weeks to provide the desired whitening effect and in some instances requires a skilled professional to apply the gel. However, it is in many situations it is desirable to cosmetically improve the appearance of the teeth, that is whiten the teeth rapidly and temporarily as in immediate anticipation of an important public appearance or social event.

There is a continuing need for a rapid temporary tooth whitening system which can be safely applied by the wearer and without the need for specially trained dental personnel. The tooth whitening to be achieved should be easily applied to the teeth and once applied to the teeth, be able to resist removal by mastication or solvating by foods and beverages for a period for at least 5 to 8 hours, but should be able to be easily removed by a wearer without harming the tooth. In this respect the prior art discloses tooth whitening coatings which can be applied to the teeth as a beauty aid providing immediate temporary whitening when applied to dental enamel in the same manner as one would apply a nail polish as for example as disclosed in U.S. Pat. Nos. 4,021,915; 4,032,627; 4,482,535 and 6,210,163, however none of the prior art temporary whitening compositions impart a “natural” look to the users teeth.

### SUMMARY OF THE INVENTION

In accordance with the present invention, there is provided a composition which is adherent to dental enamel which imparts an immediate discernable whitening effect to the enamel when applied thereto which composition is comprised of a combination of a solvent for the adhesive.

Hydroxyapatite has the same physical structure as tooth enamel as has a strong affinity to the tooth enamel surfaces with the result that the hydroxyapatite imparts a “natural” white appearance to the enamel surface in contrast to other white pigment powders such as titanium dioxide, aluminum oxide and zinc oxide which impart an unnatural appearance to the tooth enamel.

### DESCRIPTION OF PREFERRED EMBODIMENTS

Volatile Carrier

Suitable carriers for use in the preparation of the whitening compositions of the present invention include volatile solvents such water, ethanol and ethyl acetate and non-volatile, water soluble solvents such as triacetin. Generally the solvent in which the hydroxyapatite is dispersed is present in the composition at a concentration of about 10 to about 80% by weight and preferably about 40 to about 70% by weight.

Hydroxyapatite

The hydroxyapatite whitening agent used in the practice of the present invention is a calcium phosphate salt having the chemical formula Ca₅(OH)(PO₄)₃. The preparation of hydroxyapatite is well known to the art as disclosed in U.S. Pat. No. 4,274,879; U.S. Pat. No. 4,330,514; U.S. Pat. No. 4,324,772; U.S. Pat. No. 4,408,300; U.S. Pat. No. 4,097,935; U.S. Pat. No. 4,207,300. The hydroxyapatite whitening agent is present in the whitening composition of the present invention at a concentration of about 0.5 to about 60% by weight and preferably about 15 to about 40% by weight.

The whitening composition of the present invention can be applied to tooth enamel by any suitable means as for example as a flexible film or strip or as a paint on liquid.

Organic/Silicone Adhesive

The rapid tooth whitening composition according to the invention contains an organic adhesive dispersed or dissolved in the volatile solvent. Organic polymers useful as adhesion enhancing agents useful in the practice of the present invention include polymers such as Carbomers such as carboxymethylene polymers such as acrylic acid polymers and acrylic acid copolymers. Carboxypolymethylene is a slightly acidic vinyl polymer with active carboxyl groups. A carboxypolymethylene preferred for use in the practice of the present invention is a copolymer of acrylic acid cross linked with approximately 0.75% to approximately 1.5% polyallyl sucrose that is sold under the trade designation Carbopol 934, 974 by B.F. Goodrich. The adhesive is preferably a natural resin, particularly a resin selected from shellac resins, colophonium resins and modified colophonium resins. Another suitable group of adhesives is organic polymeric compositions represented by the group of alkyd resins, polyvinyl acetaldehydes, polyvinyl alcohols, polyvinyl acetates, poly(ethylene oxide), polyacrylates, ketone resins, polyvinylpyrolidone, polyvinylpyrolidone/vinyl acetate copolymer, polyethylene glycols of 200 to 1000 molecular weight and polyoxyethylene/polyoxopropylene block copolymers (Polyox), silicone resins and silicone based pressure sensitive adhesives such as those available from Dow Corning Company under the trade designation BIO-PSA.

The pressure sensitive adhesive (PSA) are well known in the art and many are commercially available. Generally, silicone based PSA's are produced by condensing a silicone resin and an organosiloxane such as a polydiorganosilioxane. the PSA is an elastomeric, tacky material, adhesion of which dental enamel surfaces can be varied by altering the ratio of silicone resin to polydiorganosiloxane in the copolymer molecule. For example PSAs available from the Dow-Corning Company under the brand name BIO-PSA is a pressure sensitive adhesive specifically designed for pharmaceutical use and is permeable to many drug compounds and finds application for the transdermal application of these compounds. The BIO-PSA silicone polymers are the copolymer product of mixing a silanol terminated polydiorganosiloxane such as polydimethyl siloxane with a silanol-containing silicone resin whereby the silanol groups of the polydiorganosiloxane undergo a condensation reaction with the silanol groups of the silicone resin so that the polydiorganosiloxane is lightly cross linked by the silicone resin (that is, the polydiorganosiloxane chains are bonded together through the resin molecules to give chain branching and entanglement and/or a small amount of network character) to form the silicone pressure sensitive adhesive. A catalyst, for example an alkaline material such as ammonia, ammonium hydroxide or ammonium carbonate can be mixed with the silanol-terminated polydiorganosiloxane and the silicone resin to promote this cross linking reaction.

By copolymerizing the silicone resin with the silanol terminated polydiorganosiloxane, the self adhering property and the cohesive properties of a soft elastomer matrix characteristic of pressure sensitive polymers and distinguished from the hard, non-elastomeric properties of silicone resins.

Modifying the silicone resin to polydiorganosiloxane ratio of the pressure sensitive adhesive will modify the tackiness of the PSA. For example, the BIO PSA silicone adhesive sold by Dow-Corning is available in three silicone resin to silicone polymer ratios namely, 65/35 (low tack), 60/40 (medium tack), 55/45 (high tack) dissolved in either ethyl acetate solvent or other solvents.

The proportion of adhesive agent present in the compositions of the present invention ranges from about 0.1% to about 99% by weight and preferably from about 20 to about 50% by weight of the composition.

Sweetening Agent

A sweetening material may be employed in the tooth whitening composition of the present invention. Suitable sweetening agents include sodium saccharin, sodium cyclamate, xylitol, aspartame and the like, in concentrations of about 0.01 to about 1% by weight and preferably about 0.05 to about 0.75% by weight. Sodium saccharin is preferred.

Flavor

The tooth whitening composition of the present invention may also contain a flavoring agent. Flavoring agents that are used in the practice of the present invention include essential oils as well as various flavoring aldehydes, esters, alcohols, and similar materials. Examples of the essential oils include oils of spearmint, peppermint, wintergreen, sassafras, clove, sage, eucalyptus, marjoram, cinnamon, lemon, lime, grapefruit, and orange. Also useful are such chemicals as menthol, carvone, and anethole. Of these, the most commonly employed are the oils of peppermint, spearmint and wintergreen. The flavoring agent is incorporated in the whitening liquid composition of the present invention at a concentration of about 0.1 to about 2% by weight and preferably about 0.1 to about 0.5% by weight.

The whitening composition of the present invention may be applied to tooth enamel by any suitable means as for example in extruded form as a strip or as a liquid such as a paint-on liquid.

Strip

The hydroxyapatite whitening composition of the present invention may be in the form of a strip prepared using a conventional solvent casting process. For example, to prepare a strip by solvent casting, the adhesive polymer or resin such as colophonium and/or polyvinylpyrolidone is dissolved in a sufficient amount of a compatible solvent such as ethanol. After a solution has been formed, the addition of the hydroxyapatite whitening agent in powder form follows and any other ingredients such as sweeteners or flavors. The solution is coated onto a suitable casting carrier material from which the formed strip can be easily released from without damage. The carrier material must have a surface tension which allows the solution to spread evenly across the intended carrier width without soaking in to form a destructive bond between the two substrates. Examples of suitable carrier materials include glass, stainless steel, teflon, polyethylene impregnated kraft paper. The strip may be dried to a solid usable form in a high temperature air bath using a drying oven, drying tunnel, vacuum drier, or any other suitable drying equipment at a temperature. Thereafter the strip formed on the carrier is peeled off the carrier surface and cut into pieces of suitable size and shape for consumer use and packed into a suitable container.

To use the whitening strip for the present invention, the strip when applied to the teeth surface will adhere to the teeth in an appropriate manner and within 1 to 60, minutes, the teeth surfaces will whiten to a natural appearance as the hydroxyapatite present in the strip matrix migrates to the tooth surfaces. In this regard, the whitening strip is formed to have a width dimension suitable to cover a row of teeth (upper or lower). Therefore, the whitening strip may be applied to the upper set of teeth, or to the lower set of teeth either separately or simultaneously. The length dimension of the whitening strip is determined by the amount of coverage desired. In this regard, the number of teeth which it is desired to whiten will determine the dimensions for the whitening strip. For instance, it may be desired to only whiten the front teeth, which are most easily seen by others. Accordingly, the length of whitening strip can be reduced in this case, as compared to the case where it is desired to whiten all of the teeth. The duration of application of whitening strip to the teeth will depend upon the type and concentration of the hydroxyapatite whitening agent, as well as the type and intensity of stain. The applied layer of hydroxyapatite will resist solvation removal by beverages or mastication for at least 2 to 8 hours and generally 3 to 5 hours. After the teeth are whitened to the satisfaction of the user, the portions of the strip can be easily removed by rinsing the mouth with water and brushing.

Paint-On Tooth Whitener

The paint on whitening composition of the present invention is prepared in the form of a flowable viscous liquid suspension containing the hydroxyapatite whitening agent and is applied as such to the users teeth as by painting the teeth with a soft applicator brush in the same manner as one would apply nail polish to a finger nail and without the intervention of a dentist or technological operations. Application by the user and evaporation or dissolution of the solvent leaves an adherent natural appearing white coating of hydroxyapatite on the teeth which provides almost instantaneous whitening of the tooth enamel.

Other means of application of the whitening composition of the present invention includes applying an adhesive strip having the hydroxyapatite whitening agent incorporated therein on the surface of an inert substrate such as polyethylene.

The layer of tooth whitening composition applied to tooth enamel contains no ingredients imparting thereto an unacceptable taste or texture, rendering it unpleasant to the user and adheres strongly to tooth enamel. The composition is sufficiently adherent to tooth enamel to remain on the teeth for the applied whitening composition enabling the applied coating to resist the forces commonly applied by the lips and tongue as well a those forces encountered during normal mastication, as upon the evaporation or dissolution of the solvent in mouth after application a hard coating of hydroxyapatite forms in about 1 minute which coating is bonded securely to the tooth enamel to which it is applied. While the layer of applied paint-on whitening composition is in place, the user is to refrain from mastication. The whitening composition can be removed as and when required, at will, by an employment of standard oral hygiene procedures such as brushing or by rinsing with an alcoholic mouthwash.

The whitening composition of the present invention is prepared by adding the ingredients of the composition in a suitable vessel such as a stainless steel tank provided with a mixer. In the preparation for the whitening composition, the ingredients are advantageously added to the mixer in the following order: solvent, adhesive materials, hydroxyapatite and any desired flavoring or sweetener. The ingredients are then mixed to form a homogeneous dispersion. When mixed and ready for extrusion or application directly as a paint-on whitener, the composition is a white colored opaque viscous liquid composition.

Other ingredients which may be incorporated in the composition of the present invention include any of the materials commonly used in the oral care formulations. These include: antimicrobial agents, e.g., Triclosan, chlorhexidine, copper-, zinc- and stannous salts such as zinc citrate, zinc sulphate, zinc glycinate, sodium zinc citrate and stannous pyrophosphate, sanguinarine extract, metronidazole, quaternary ammonium compounds, such as cetylpyridinium chloride; bis-guanides, such as chlorhexidine digluconate, hexetidine, octenidine, alexidine; and halogenated bisphenolic compounds, such as 2,2′methylenebis-(4-chloro-6-bromophenol); antiinflammatory agents such as ibuprofen, flurbiprofen, aspirin, indomethacine etc.; anticaries agents such as sodium-, calcium-, magnesium- and stannous fluoride, aminefluorides, disodium monofluorophosphate and sodium trimeta phosphate; plaque buffers such as urea, calcium lactate, calcium glycerophosphate and strontium polyacrylates; vitamins such as Vitamin C; plant extracts; desensitizing agents, e.g., potassium citrate, potassium chloride, potassium tartrate, potassium bicarbonate, potassium oxalate, potassium nitrate and strontium salts; agents effective against dental calculus such as pyrophosphate salts including the mono, di, tri and tetra alkali metal and ammonium pyrophosphate and tripolyphosphate salts; biomolecules, e.g., bacteriocins, antibodies, enzymes such as papain, glucoamylase; opacifying agents, pigments, coloring agents and fluoride ion providing salts having anticaries efficacy such as sodium fluoride, potassium fluoride, a tin fluoride such as stannous fluoride.

The present invention is illustrated by the following example but is not to be limited thereby.

### EXAMPLE

A series of hydroxyapatite whitening strips were prepared having the ingredients listed in Table I below.

TABLE I

| Strip Composition | A | B | C | D | E |
|---|---|---|---|---|---|
| Ethanol | 35.73 | 31.02 | 35.73 | 35.73 | 0 |
| Ethylacetate | 0 | 0 | 0 | 0 | 37.7 |
| Polyvinylpyrrolidone/ | 8.91 | 8.68 | 0 | 0 | 6.0 |
| vinyl acetate | | | | | |
| Polyvinylpyrrolidone | 0 | 0 | 8.91 | 0 | 2.9 |
| Hydroxyapatite | 19.62 | 24.32 | 19.62 | 19.62 | 19.6 |
| Colophonium | 35.74 | 31.02 | 35.74 | 35.74 | 31.7 |
| PEG 600 | 0 | 4.96 | 0 | 0 | 3.0 |
| Polyox | 0 | 0 | 0 | 7.0 | 0 |
| Water | 0 | 0 | 0 | 1.91 | 0 |
| Total (%) | 100.00 | 100.00 | 100.00 | 100.00 | 100.00 |

The strip compositions of Table I were prepared by slowly adding the adhesive material (such as colophonium, polyvinyl pyrolidone, polyvinyl pyrolidone/vinyl acetate, Polyox, PEG 600 or mixtures thereof) into the solvent (ethanol, ethyl acetate) with vigorous stirring to form a translucent and viscous solution. To this solution was added hydroxyapatite powder and thoroughly mixed. The resulting white mixture was cast onto a polyethylene coated kraft paper and allowed to dry overnight. A white colored strip was formed which was readily peeled off from the plate. The dry strip had a thickness of about 0.1 to 0.25 mm. The whitening efficacy of the strips was examined by wetting and placing piece of the strip (13 mm times 100 mm weighing 150-500 milligrams (mg) against the tooth surface of subjects who had stained teeth, and the time recorded for the teeth to assume a natural white appearance was recorded. The results are recorded in Table II below.

TABLE II

| Composition | Time to Whiten Teeth (minutes) |
|---|---|
| A | 20 |
| B | 15 |
| C | 12 |
| D | 10 |
| E | 5 |

After application the film that formed on the teeth imparted a natural white appearance to the teeth and remained on the teeth for 3 to 8 hours before removal was attempted.

### EXAMPLE II

A series of paint-on whitening compositions were prepared containing the ingredients listed in Table III below.

TABLE III
Liquid Paint-On Formulation

| | F | G | H | I | J | K |
|---|---|---|---|---|---|---|
| BIO-PSA* | 50 | 40 | 25 | 50 | 25 | 34.9 |
| Ethylacetate | 33 | 48 | 48 | 20 | 45 | 40 |
| Mineral Oil | 2 | 2 | 2 | — | 2 | — |
| Acetin | — | — | — | 10 | — | — |
| Hydroxyapatite | 5 | 10 | 25 | 20 | 25 | 25 |
| Carbopol 974 | — | — | — | — | 3 | 0.1 |

*silicone polymer based pressure sensitive adhesive

The compositions were painted on tooth surfaces and imparted a natural white appearance to the teeth for 3 to 8 hours before removal was attempted.

1. A composition for the rapid, temporary whitening of teeth comprised of an adhesive agent and hydroxyapatite which composition imparts a natural white appearance to dental enamel when applied thereto.

2. The composition of claim 1 wherein the composition contains 0.1 to 99% by weight of the adhesive, and about 0.5 to about 60% by weight of calcium hydroxyapatite.

3. The composition as recited in claim 1, wherein said adhesive is selected from the group consisting of natural resins, modified natural resins and pressure sensitive adhesives.

4. The composition as recited in claim 1 wherein said adhesive is selected from polyvinylpyrolidone, polyvinylpyrolidone/vinyl acetate copolymers and polyethylene glycol.

5. The composition of claim 1 which is in the form of a strip that can be applied to and adheres to dental enamel.

6. The composition of claim 1 which is in the form of a paint-on liquid dispersion.

7. A method for tooth whitening comprising the steps of:
a) applying a composition as defined in claim 1 to the surface of to the enamel; and,
b) allowing the composition to dry on said surface.
